SEQUENCE LISTING (20 sequences)

SEQ ID NO: 1 - DNA, 2724 bp, Artificial sequence
  synthesized
ctagatctacgccaggaccgagcaagcccagatgagaaccgacgcagatttccttggcacctgttgcttcagctgaatcctggcaatacgagatacctgctttgaatattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccag

SEQ ID NO: 2 - DNA, 997 bp, Artificial sequence
  synthesized
gccattggcggaggggtccggacggtcaggaacttagccttatgagatgaatgatggacgtgtctggcctcggaaaaggatatatggggatcatgatagtactagccatattaatgaagggcatataccacgcgttggacctgcgttatagcttcccgttagttatagtaccatcgttataccagccaatcaagtcaccacgcacgaccggggacggcgaatccccgggaattgaaagaaattgcatcccaggccagtgaggccagcgattggccacctctccaaggcacagggccattctgcagcgctggtggattcatcgcaatttcccccggcccggcccgacaccgctataggctggttctcccacaccatcggagattcgtcgcctaatgtctcgtccgttcacaagctgaagagcttgaagtggcgagatgtctctgcaggaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacacctcagcacttaagactagtacgcgtctcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcat

SEQ ID NO: 3 - DNA, 814 bp, Artificial sequence
  synthesized
atgttgtaccttcctctcgttgccctctcctttgctgccaccgttcctctggtgagcgcataccccatcactggcgatggtgtcaactgccgctctggtcctggcaccaaccatgccgtggtcaagtcttaccccaagggccatgagatctccattgtctgccaggctgccggcaccaacgtcaagggagatgagctctgggacaagacgtccgacggctgctatgtcgccgattactacgtgaagaccggtaccactggctatgtcaccaagcactgcgatggcggcagtgatggtggcagcggcggcggcagcggcaatcttcccggtctcactgccactcagtcctctcacgctcatgcaatcatcggtgaagcaaagaaggaaggcctgggtcgtcaaggctgtctggctggtattgcaactggcttggtcgaggtgagtacctattcctcttcattgcatcggcaattatgatcattgtactgatggtgagctattatagtccaatcttttgatctatgccaatagcaaggtacccgagtcgctcaaataccaccatgatgccgtcggccacgactacgacagcgtgggcatcttccagcaacgtgctgtctactaccccaacatcgctgctgacatggaccctgcacgctctgcggctcagttctttgccaagatgaagaatatcagcggctggaagacgatggatgtcggcaagctgtgccagaaggtgcaggtctccgcctaccccgatcggtatgcgcaacgtgtgcctgctgctgagaagatctgcgctgctggtggactgtag

SEQ ID NO: 4 - protein, 248 aa, Artificial sequence
  synthesized
MLYLPLVALSFAATVPLVSAYPITGDGVNCRSGPGTNHAVVKSYPKGHEISIVCQAAGTNVKGDELWDKTSDGCYVADYYVKTGTTGYVTKHCDGGSDGGSGGGSGNLPGLTATQSSHAHAIIGEAKKEGLGRQGCLAGIATGLVESNLLIYANSKVPESLKYHHDAVGHDYDSVGIFQQRAVYYPNIAADMDPARSAAQFFAKMKNISGWKTMDVGKLCQKVQVSAYPDRYAQRVPAAEKICAAGGL

SEQ ID NO: 5 - DNA, 838 bp, Artificial sequence
  synthesized
atgcagctctccctcctcgtcctctccctcgtggccgctgtgcccatggccagcgcgtacccggtcaaggccgacactctcaactgccgctccggcccgggcaccagttacaaggtcatcaagacctacaagaagggcaccgatctcaagatcacctgccagacgcccggcacctcggtcaacggcgacaacctgtgggacaagacctcggacggctgctacgtggccgattactacgtcaagaccggcacctccggctacgtcacggcccattgcgatgccggcagcggcagcggcagcagcggcggcggcaacctgccaggactcaactcggtccagtcctcgcacgcccgggccatcatcggcgaggcgaagaaggagggcgtcggccgccacggctgcgaggccggcatcgcgaccgcgcttgtcgaggtacgttgcatcctaacatcaacacttacttgccttgaccccactgtcaccgccagaaaaaaccaaaactaacacatcacctcttcccctcacacagtccaacatcctgatctacgccaacaaggcggtcccggcctcgctcaagtacccgcacgacgcggtgggctcggaccacgacagcgtcggcatcttccagcagcgcgccaagtactaccccaacatcgcggccgacatggacccggcgcgctcggccgcccagttcttcgccaagatgaagggcatcaagggctggcagagcatggccgtcggcacgctctgccagaaggtccagggctccgcgtacccggaccgctatgccaagcgggtctcggaggcgaccaagatttgccaggctggtgggttgtaa

SEQ ID NO: 6 - protein, 246 aa, Artificial sequence
  synthesized
MQLSLLVLSLVAAVPMASAYPVKADTLNCRSGPGTSYKVIKTYKKGTDLKITCQTPGTSVNGDNLWDKTSDGCYVADYYVKTGTSGYVTAHCDAGSGSGSSGGGNLPGLNSVQSSHARAIIGEAKKEGVGRHGCEAGIATALVESNILIYANKAVPASLKYPHDAVGSDHDSVGIFQQRAKYYPNIAADMDPARSAAQFFAKMKGIKGWQSMAVGTLCQKVQGSAYPDRYAKRVSEATKICQAGGL

SEQ ID NO: 7 - DNA, 844 bp, Artificial sequence
  synthesized
atgttgtaccttcctctcgttgccctctcctttgctgccaccgttcctctggtgagcgcataccccatcactggcgatggtgtcaactgccgctctggtcctggcaccaaccatgccgtggtcaagtcttaccccaagggccatgagatctccattgtctgccaggctgccggcaccaacgtcaagggagatgagctctgggacaagacgtccgacggctgctatgtcgccgattactacgtgaagaccggtaccactggctatgtcaccaagcactgcgatggcggcagtgatggtggcagcggcggcggcagcggcaatcttcccggtctcaactcggtccagtcctcgcacgcccgggccatcatcggcgaggcgaagaaggagggcgtcggccgccacggctgcgaggccggcatcgcgaccgcgcttgtcgaggtacgttgcatcctaacatcaacacttacttgccttgaccccactgtcaccgccagaaaaaaccaaaactaacacatcacctcttcccctcacacagtccaacatcctgatctacgccaacaaggcggtcccggcctcgctcaagtacccgcacgacgcggtgggctcggaccacgacagcgtcggcatcttccagcagcgcgccaagtactaccccaacatcgcggccgacatggacccggcgcgctcggccgcccagttcttcgccaagatgaagggcatcaagggctggcagagcatggccgtcggcacgctctgccagaaggtccagggctccgcgtacccggaccgctatgccaagcgggtctcggaggcgaccaagatttgccaggctggtgggttgtaa

SEQ ID NO: 8 - protein, 248 aa, Artificial sequence
  synthesized
MLYLPLVALSFAATVPLVSAYPITGDGVNCRSGPGTNHAVVKSYPKGHEISIVCQAAGTNVKGDELWDKTSDGCYVADYYVKTGTTGYVTKHCDGGSDGGSGGGSGNLPGLNSVQSSHARAIIGEAKKEGVGRHGCEAGIATALVESNILIYANKAVPASLKYPHDAVGSDHDSVGIFQQRAKYYPNIAADMDPARSAAQFFAKMKGIKGWQSMAVGTLCQKVQGSAYPDRYAKRVSEATKICQAGGL

SEQ ID NO: 9 - DNA, 23 bp, Artificial sequence
  synthesized
cttggcgtaatcatggtcatagc

SEQ ID NO: 10 - DNA, 40 bp, Artificial sequence
  synthesized
cggacccctccgccaatggccttgcatgcaggcctctgca

SEQ ID NO: 11 - DNA, 20 bp, Artificial sequence
  synthesized
ctagatctacgccaggaccg

SEQ ID NO: 12 - DNA, 40 bp, Artificial sequence
  synthesized
atgaccatgattacgccaagcttctggaaacgcaaccctg

SEQ ID NO: 13 - DNA, 20 bp, Artificial sequence
  synthesized
gccattggcggaggggtccg

SEQ ID NO: 14 - DNA, 40 bp, Artificial sequence
  synthesized
cggtcctggcgtagatctagatgcattgaatgacagtgat

SEQ ID NO: 15 - DNA, 52 bp, Artificial sequence
  synthesized
catccccagcatcattacacctcagcaatgttgtaccttcctctcgttgccc

SEQ ID NO: 16 - DNA, 55 bp, Artificial sequence
  synthesized
aggtgtcagtcaccctctagatctcgagctacagtccaccagcagcgcagatctt

SEQ ID NO: 17 - DNA, 48 bp, Artificial sequence
  synthesized
catccccagcatcattacacctcagcaatgcagctctccctcctcgtc

SEQ ID NO: 18 - DNA, 52 bp, Artificial sequence
  synthesized
gtgtcagtcaccctctagatctcgagttacaacccaccagcctggcaaatct

SEQ ID NO: 19 - DNA, 808 bp, Artificial sequence
  synthesized
atgcagctctccctcctcgtcctctccctcgtggccgctgtgcccatggccagcgcgtacccggtcaaggccgacactctcaactgccgctccggcccgggcaccagttacaaggtcatcaagacctacaagaagggcaccgatctcaagatcacctgccagacgcccggcacctcggtcaacggcgacaacctgtgggacaagacctcggacggctgctacgtggccgattactacgtcaagaccggcacctccggctacgtcacggcccattgcgatgccggcagcggcagcggcagcagcggcggcggcaacctgccaggactcactgccactcagtcctctcacgctcatgcaatcatcggtgaagcaaagaaggaaggcctgggtcgtcaaggctgtctggctggtattgcaactggcttggtcgaggtgagtacctattcctcttcattgcatcggcaattatgatcattgtactgatggtgagctattatagtccaatcttttgatctatgccaatagcaaggtacccgagtcgctcaaataccaccatgatgccgtcggccacgactacgacagcgtgggcatcttccagcaacgtgctgtctactaccccaacatcgctgctgacatggaccctgcacgctctgcggctcagttctttgccaagatgaagaatatcagcggctggaagacgatggatgtcggcaagctgtgccagaaggtgcaggtctccgcctaccccgatcggtatgcgcaacgtgtgcctgctgctgagaagatctgcgctgctggtggactgtag

SEQ ID NO: 20 - protein, 246 aa, Artificial sequence
  synthesized
MQLSLLVLSLVAAVPMASAYPVKADTLNCRSGPGTSYKVIKTYKKGTDLKITCQTPGTSVNGDNLWDKTSDGCYVADYYVKTGTSGYVTAHCDAGSGSGSSGGGNLPGLTATQSSHAHAIIGEAKKEGLGRQGCLAGIATGLVESNLLIYANSKVPESLKYHHDAVGHDYDSVGIFQQRAVYYPNIAADMDPARSAAQFFAKMKNISGWKTMDVGKLCQKVQVSAYPDRYAQRVPAAEKICAAGGL

(19) United States
(12) Patent Application Publication
(10) Pub. No.: US 2024/0093171 A1
(43) Pub. Date: Mar. 21, 2024

(54) CHIMERIC LYSOZYME VARIANT AND APPLICATION THEREOF IN ANIMAL FEED ADDITIVE

(71) Applicants: NANJING BESTZYME BIO-ENGINEERING CO., LTD., Nanjing, Jiangsu (CN); JINAN BESTZYME BIO-ENGINEERING CO., LTD., Jinan, Shandong (CN)

(72) Inventors: Fei ZHENG, Nanjing, Jiangsu (CN); Ting YAN, Nanjing, Jiangsu (CN); Jidong ZHU, Nanjing, Jiangsu (CN); Hong XU, Nanjing, Jiangsu (CN); Yan SUN, Nanjing, Jiangsu (CN); Yanjie LIU, Jinan, Shandong (CN)

(21) Appl. No.: 18/254,428

(22) PCT Filed: Nov. 12, 2021
(86) PCT No.: PCT/CN2021/130202

§ 371 Date: May 25, 2023

(30) Foreign Application Priority Data
Nov. 26, 2020 (CN) 202011346101.6
Apr. 28, 2021 (CN) 202110464931.7

(51) Int. Cl. C12N 9/36; A23K 20/189

(52) U.S. Cl. CPC C12N 9/2462; A23K 20/189; C12Y 302/01017

(57) ABSTRACT

A chimeric lysozyme variant has an improved heat resistance and is encoded by a polynucleotide. A novel chimeric lysozyme sequence is constructed, and high-efficiency expression in a host cell is achieved. The thermal stability of the chimeric lysozyme under the condition of 60° C. is nearly doubled compared to a parental sequence. The chimeric lysozyme variant can be used to prepare product that is antibiotic, for example, animal feed additive containing the chimeric lysozyme variant and an animal feed containing the animal feed additive.

### TECHNICAL FIELD

The present invention falls within the technical field of bioengineering, and relates to a chimeric lysozyme variant and an application thereof in an animal feed additive.

### BACKGROUND ART

Lysozyme (EC 3.2.1.17) is a hydrolytic enzyme acting on the cell wall of microorganisms, also known as muramidase. It can effectively hydrolyze the peptidoglycan of the bacterial cell wall, which is mainly achieved by the mechanism of hydrolysis of the β-1,4 glycosidic bond between N-acetylmuramic acid and N-acetylglucosamine to break the peptidoglycan backbone structure, resulting in cell wall rupture and eventually bacteriolysis.

Lysozyme naturally exists in many organisms, such as viruses, plants, insects, birds, reptiles and mammals. In mammals, lysozyme has been isolated from nasal secretions, saliva, tears, intestinal contents, urine and milk. At present, lysozyme has been classified into seven different glycoside hydrolase families (CAZy www.cazy.org): GH18, GH19, egg white lysozyme (GH22), goose egg white lysozyme (GH23), bacteriophage T4 lysozyme (GH24), Sphingomonas flagellin (GH73) and Chalaropsis lysozyme (GH25). Lysozyme is a non-toxic protein that has no side effects on humans and mammals. It has been widely used in various industries in recent years due to its bacteriolytic properties. Lysozyme can be used as a natural preservative in dairy industry. For example, adding lysozyme to pasteurized milk can effectively prolong its shelf life. In food applications, adding lysozyme can prolong the storage time of aquatic products and meat foods. In animal feed industry, the long-term and considerable use of antibiotics has had serious negative effects, manifested in increasingly serious drug resistance in livestock and poultry, and the emergence of a large number of highly drug-resistant strains and even new pathogenic strains, which not only makes the prevention and control of animal epidemic diseases more difficult, but also seriously affects the quality and safety of livestock and poultry products and directly threatens human health. The international community has recognized the harm of antibiotics, and many countries have begun to prohibit the addition of antibiotics to livestock and poultry feeds. In this context, lysozyme, as a non-specific immune factor, has an important application prospect in alternatives to antibiotics. Lysozyme can catalyze the hydrolysis of the β-1,4 glycosidic bond between N-acetylmuramic acid and N-acetylglucosamine in the bacterial cell wall, which leads to the exudation of bacterial contents and the lysis of bacteria, thus achieving effects such as antibacterial. Moreover, lysozyme can effectively decompose the pus of injured tissues and enhance the defense function, thus effectively protecting the digestive tract lining and accelerating the repair of the intestinal tract and injured tissues. In addition, lysozyme used in combination with polyphosphate and glycine has a good preservative effect. Adding lysozyme to feeds can prevent mildew, prolong the storage period of feeds and reduce unnecessary losses. The combination of lysozyme and glucose oxidase also provides a synergistic effect.

Currently, the application of lysozyme in feed and breeding production industries is greatly limited. The main reasons are as follows: one is that, currently, a significant proportion of the lysozyme is extracted from egg white, resulting in low productivity and output, as well as high costs; the other is that, most of the lysozyme has poor thermal stability and cannot meet the process requirements of high-temperature granulation of feeds. Therefore, it is of great significance to research and develop new lysozyme products obtained by microbial fermentation with lower cost and improved thermal stability.

### SUMMARY OF THE INVENTION

To solve the above-mentioned technical problems, an objective of the present invention is to provide a chimeric lysozyme variant having improved heat resistance and a polynucleotide encoding same. A novel chimeric lysozyme variant sequence is constructed, and high-efficiency expression thereof in a host cell is achieved. The thermal stability of the chimeric lysozyme variant under the condition of 60° C. is nearly doubled compared to a parental sequence. Another objective of the present invention is to provide an application of a chimeric lysozyme variant having improved heat resistance in an animal feed additive. The feed additive has a significant effect on animal growth performance and greatly improves the conversion rate of the feed.

The present invention provides a chimeric lysozyme variant, wherein the chimeric lysozyme variant has an amino acid sequence set forth in SEQ ID NO: 8, or having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 8; and the chimeric lysozyme variant has a lysozyme activity.

In a preferred embodiment, the amino acid sequence is set forth in SEQ ID NO: 8, that is, the amino acid sequence is obtained by substituting the first 109 amino acids at the N-terminus of the amino acid sequence set forth in SEQ ID NO: 6 with the first 111 amino acids at the N-terminus of the amino acid sequence set forth in SEQ ID NO: 4.

The present invention also provides the use of the chimeric lysozyme variant in the preparation of a product as an alternative to an antibiotic. The “product as an alternative to an antibiotic” refers to a product that can substitute an antibiotic, which can be used in an animal feed additive as a potential substitute for an antibiotic to inhibit the reproduction of harmful microorganisms, protect the animal intestinal health, and improve the animal immunity.

The present invention also provides the use of the chimeric lysozyme variant as described above, wherein the use is:
in an animal feed;
in an animal feed additive;
in the preparation of a composition for use in an animal feed;
for improving the nutritional value of an animal feed;
for improving the digestibility of an animal feed; and/or
for improving one or more performance parameters of an animal.

The present invention also provides a method for improving the nutritional value of an animal feed, comprising adding the chimeric lysozyme variant as described above to the feed.

The present invention also provides an animal feed additive, which comprises the chimeric lysozyme variant as described above.

Further, the feed additive comprises one or more components selected from the group consisting of:
one or more vitamins;
one or more minerals;
one or more amino acids;
one or more phytochemicals;
one or more prebiotics;
one or more organic acids; and
one or more other feed ingredients.

The vitamins include fat-soluble vitamins and water-soluble vitamins. Non-limiting examples of the fat-soluble vitamins include vitamin A, vitamin D3, vitamin E and vitamin K, e.g., vitamin K3. Non-limiting examples of the water-soluble vitamins include vitamin C, vitamin B12, biotin and choline, vitamin B1, vitamin B2, vitamin B6, nicotinic acid, folic acid and pantothenate, e.g., Ca-D-pantothenate.

Non-limiting examples of the minerals include calcium, magnesium, phosphorus, potassium, sodium, boron, cobalt, chloride, chromium, copper, fluoride, iodine, iron, manganese, molybdenum, selenium and zinc.

Non-limiting examples of the amino acids are lysine, alanine, beta-alanine, threonine, methionine and tryptophan.

The phytochemicals are a group of natural growth promoters or non-antibiotic growth promoters derived from herbs, spices or other plants used as feed additives. Phytochemicals can be single substances prepared from essential oils/extracts, single plants and mixtures of plants (herbal products) or mixtures of essential oils/extracts/plants (specialized products). Examples of the phytochemicals are rosemary, sage, oregano, thyme, clove and lemongrass. Examples of the essential oils are thymol, eugenol, m-cresol, vanillin, salicylate, resorcinol, guajacol, gingerol, lavender oil, ionone, irone, cineole, menthol, peppermint oil, alpha-pinene, limonene, anethole, linalool, methyl dihydrojasmonate, carvacrol, propionic acid/propionate, acetic acid/acetate, butyric acid/butyrate, rosemary oil, clove oil, geraniol, terpineol, citronellol, amyl salicylate and/or benzyl salicylate, cinnamaldehyde, plant polyphenol (tannin), turmeric and turmeric extract.

The prebiotics are substances that induce the growth or activity of microorganisms (for example, bacteria and fungi), which contribute to the well-being of their host. Prebiotics are typically non-digestible fiber ingredients that pass through the upper part of the gastrointestinal tract without being digested. They stimulate the growth or activity of beneficial bacteria that colonize in the large intestine by serving as a substrate for these bacteria. Generally, prebiotics increase the number or activity of Bifidobacteria and lactic acid bacteria in the gastrointestinal (GI) tract. Yeast derivatives (inactivated whole yeast or yeast cell wall) can also be considered as prebiotics. They generally include mannooligosaccharides, yeast glucan or protein contents, and are generally derived from the cell wall of yeast (Saccharomyces cerevisiae).

The organic acids are widely distributed in nature as normal components of plant or animal tissues. They are also formed by microbial fermentation of carbohydrates mainly in the large intestine. Generally, the organic acids are used as substitutes for antibiotic growth promoters in pig and poultry production due to their preventive effect on intestinal problems such as necrotic enteritis in chickens and Escherichia coli infection in piglets. Organic acids can be sold as a single component or generally as a mixture of two or three different organic acids. Examples of the organic acids are propionic acid, formic acid, citric acid, lactic acid, sorbic acid, malic acid, acetic acid, fumaric acid, benzoic acid, butyric acid and tartaric acid or salts thereof (generally sodium or potassium salts, such as potassium dicarboxylate or sodium butyrate).

The feed additive of the present invention may further comprise colorants, stabilizers, growth improving additives and aromatic compounds/flavorings, polyunsaturated fatty acids (PUFAs), reactive oxygen generating substances, antioxidants, antimicrobial peptides, antifungal polypeptides and mycotoxin control compounds.

In a preferred embodiment, the chimeric lysozyme variant is added in an amount of 100-1000 g chimeric lysozyme variant per ton of animal feed (100-1000 g/t); preferably, the chimeric lysozyme variant is added in an amount of 250-500 g chimeric lysozyme variant per ton of animal feed (250-500 g/t).

The present invention also provides an animal feed containing the animal feed additive as described above.

Further, the feed contains basal diet.

The basal diet is formulated separately according to different growth stages as guided by Nutritional Requirements of Chicken of NRC (1994), Feeding Standard of Chicken in China (2004) and Tables of Feed Composition and Nutritive Values in China (2020).

In a preferred embodiment, the animal feed additive is added in an amount of 250-500 g animal feed additive per ton of animal feed (250-500 g/t).

Lysozyme is a non-toxic natural protein, and is a highly safe feed additive. Without any antibiotics, the addition of a lysozyme preparation to a feed can promote the digestion and absorption of nutrients by animals, improve the weight gain and feed conversion rate of animals, and reduce the morbidity and mortality of animals.

The present invention also provides a polynucleotide encoding the chimeric lysozyme variant as described above.

In a preferred embodiment, the polynucleotide has a nucleotide sequence set forth in SEQ ID NO: 7.

The present invention also provides a recombinant vector containing the polynucleotide encoding the chimeric lysozyme variant as described above.

The present invention also provides a host cell containing the recombinant vector as described above.

The host cell can be any of the Aspergillus niger, Pichia pastoris, Aspergillus oryzae, Trichoderma reesei, Bacillus subtilis, Bacillus licheniformis or Escherichia coli. The present invention has no specific limitations on the types of the host cell, so long as the chimeric lysozyme variant of the present invention can be successfully expressed by conventional experimental methods.

The present invention also provides a method for shake flask culture of a recombinant expression strain formed by lysozyme and Aspergillus niger, comprising inoculating a shake flask containing a YPM culture medium with lysozyme positive transformants, culturing same on a shaker and centrifuging same to collect a supernatant of the fermentation broth, and determining the enzyme activity of the lysozyme.

The YPM culture medium contains the following components with content in percent: yeast extract 0.2%, peptone 0.2%, and maltose 2%. The culture on a shaker is carried out at a temperature of 30-35° C.; preferably, 34° C.

The culture on a shaker is carried out at a rotating speed of 180-250 rpm; preferably, 220 rpm.

The culture on a shaker lasts for 4-6 days; preferably, 5 days.

The specific meanings of the following terms are shown below.

Sequence identity: the percent sequence identity is determined by a computer program based on a dynamic programming algorithm. Preferred computer programs within the scope of the present invention include the BLAST (Basic Local Alignment Search Tool) search program designed to explore all available sequence databases, regardless of whether the query is for protein or DNA. The BLAST version 2.0 of this search tool (Gapped BLAST) has been publicly available on the Internet (currently in http://www.ncbi.nlm.nih.gov/BLAST/). It uses an exploratory algorithm to search for local alignment instead of global alignment, enabling detection of the relationship between sequences that only share separated regions. Scores specified in the BLAST search have well-defined statistical explanations. The program preferably runs with selectable parameters set as default values.

Transformation refers to the introduction of an exogenous nucleic acid into a cell. In particular, it refers to the stable integration of a DNA molecule into the genome of a target organism.

Chimeric lysozyme variant refers to a polypeptide comprising domains from two or more polypeptides, for example, a binding domain from one polypeptide and a catalytic domain from another polypeptide. Domains can be fused at the N-terminus or C-terminus.

Lysozyme positive transformant specifically refers to a recombinant expression strain formed by transforming an expression plasmid containing a lysozyme sequence into a host strain, such as the recombinant expression strain formed by transforming the lysozyme lyzAth-amdS expression plasmid into a host Aspergillus niger as mentioned in example 2 of the present invention.

Lysozyme activity means bacteriolysis resulting from hydrolysis of the 1,4-β-bond between N-acetylmuramic acid and N-acetyl-D-glucosamine residues in peptidoglycan or between N-acetyl-D-glucosamine residues in chitodextrin. Lysozyme belongs to the class of enzymes EC3.2.1.17. The lysozyme activity is generally measured by turbidimetry, such as turbidity changes in a suspension of Micrococcus luteus CICC10680 as induced by bacteriolysis of the lysozyme. Under appropriate experimental conditions, these changes are proportional to the amount of lysozyme in a culture medium (c.f. INS 1105 of the Combined Compendium of Food Additive Specification of the Food and Agriculture Organization of the United Nations (www.fao.org)).

Heat resistance refers to the lysozyme activity after a period of incubation at an elevated temperature relative to the parental or reference sequence, either in a buffer or under conditions such as those that may be encountered during product storage/transport or conditions similar to those that exist during industrial use of the variant.

Animal feed refers to any compound, preparation or mixture suitable for or intended for intake by animals. Animal feeds for monogastric animals usually comprise concentrates as well as vitamins, minerals, enzymes, direct-fed microorganisms, amino acids and/or other feed ingredients (for example, in premixes), whereas animal feeds for ruminants usually comprises forage (including coarse grains and silage) and may further comprise concentrates as well as vitamins, minerals, enzymes, direct-fed microorganisms, amino acids and/or other feed ingredients (for example, in premixes).

The beneficial effects of the present invention are as follows. In the present invention, a novel chimeric lysozyme variant sequence is constructed, and high-efficiency expression thereof in Aspergillus niger is achieved. The heat resistance of the chimeric lysozyme variant sequence is remarkably improved compared to a parental sequence. In addition, based on the present invention, a new lysozyme product can be developed, which can bring a brand-new solution to protection of the animal intestinal health and improvement of the feed utilization rate under the background of “reducing and substituting antibiotics” in the national feed industry, and can be applied commercially.

### BRIEF DESCRIPTION OF THE DRAWINGS

FIG. 1 is a map of lysozyme lyzAth expression plasmid.

FIG. 2 is a map of lysozyme lyzTte expression plasmid.

FIG. 3 is a map of lysozyme lyzAT expression plasmid.

FIG. 4 is a map of lysozyme lyzTA expression plasmid.

FIG. 5 shows the residual enzyme activity of three lysozymes after water bath treatment at 60° C. for 3 min.

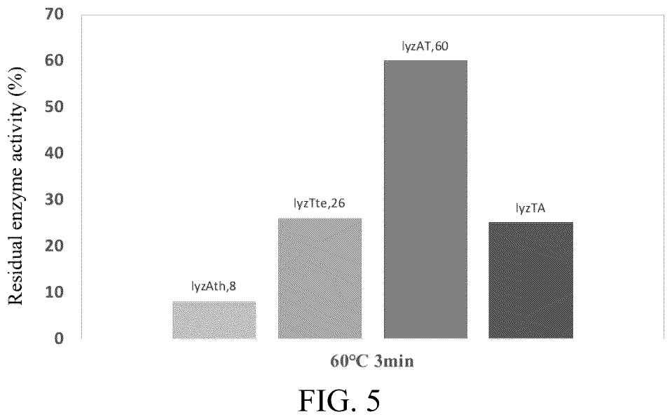

### DETAILED DESCRIPTION OF EMBODIMENTS

The present invention will be further illustrated in detail in conjunction with the following specific examples and drawings. Except for the content specifically mentioned below, the process, conditions, experimental methods, etc. used when implementing the present invention are all common knowledge and common sense in the art, and the present invention has no particular limitations on the content.

### Example 1. Construction of Lysozyme Expression Plasmid, Including the Following Parts

(1) a pUC57 plasmid was linearized with vector-F and vector-R primers;
(2) a marker amdS expression cassette was selected, which was synthesized by GenScript company, and had a sequence set forth in SEQ ID NO: 1;
(3) a DNA fragment containing the promoter and terminator of glucoamylase gene gla of Aspergillus niger was synthesized by GenScript company, and had a sequence set forth in SEQ ID NO: 2;
(4) lysozyme gene sequences:
lysozyme lyzAth (having a nucleotide sequence set forth in SEQ ID NO: 3 and an amino acid sequence set forth in SEQ ID NO: 4) derived from Aspergillus thermomutatus;
lysozyme lyzTte (having a nucleotide sequence as set forth SEQ ID NO: 5 and an amino acid sequence set forth in SEQ ID NO: 6) derived from Thermothielavioides terrestris;
the first 109 amino acids at the N-terminus of the amino acid sequence of lysozyme lyzTte derived from Thermothielavioides terrestris (set forth in SEQ ID NO: 6) were substituted with the first 111 amino acids at the N-terminus of the amino acid sequence of lysozyme lyzAth derived from Aspergillus thermomutatus (set forth in SEQ ID NO: 4) to obtain the chimeric lysozyme variant lyzAT (having a nucleotide sequence set forth in SEQ ID NO: 7 and an amino acid sequence set forth in SEQ ID NO: 8);
the first 111 amino acids at the N-terminus of the amino acid sequence of lysozyme lyzAth derived from Aspergillus thermomutatus (set forth in SEQ ID NO: 4) were substituted with the first 109 amino acids at the N-terminus of the amino acid sequence of lysozyme lyzTte derived from Thermothielavioides terrestris (set forth in SEQ ID NO: 6) to obtain the chimeric lysozyme variant lyzTA (having a nucleotide sequence set forth in SEQ ID NO: 19 and an amino acid sequence set forth in SEQ ID NO: 20);
and the above-mentioned gene sequences were synthesized by GenScript company.

Firstly, primers amdS-F and amdS-R, and gla-F and gla-R were used for PCR amplification to obtain an amdS gene with a recombinant arm and a DNA fragment containing gla promoter and terminator, respectively. The above-mentioned linearized pUC57 plasmid, amdS gene and DNA fragment containing gla promoter and terminator were recombined by using Gbson Master Mix Kit (E2611, New England Biolabs) to obtain a pGla-amdS plasmid, which was sequenced to confirm the correct sequences. The plasmid could be linearized at an AflII site and then used for insertion of the lysozyme gene.

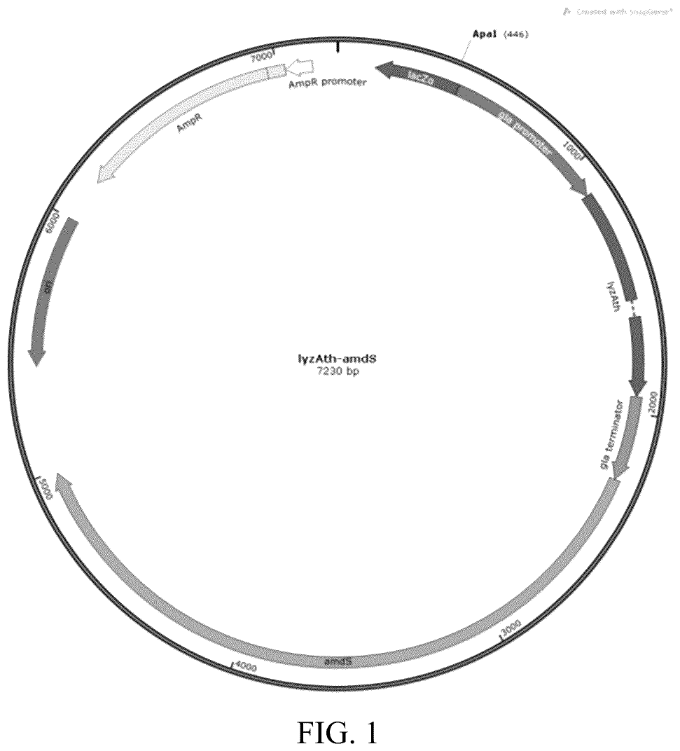

The lysozyme lyzAth expression plasmid was constructed as follows: primers lyzAth-F and lyzAth-R were used for PCR amplification to obtain a lyzAth gene with a recombinant arm, and then the lyzAth gene was recombined with the linearized pGla-amdS plasmid by using Gibson Master Mix Kit (E2611, New England Biolabs) to obtain a plyzAth-amdS plasmid, which was sequenced to confirm the sequences. The map of the constructed plasmid was as shown in FIG. 1. The plasmid could be linearized at an ApaI site and then used for protoplast transformation.

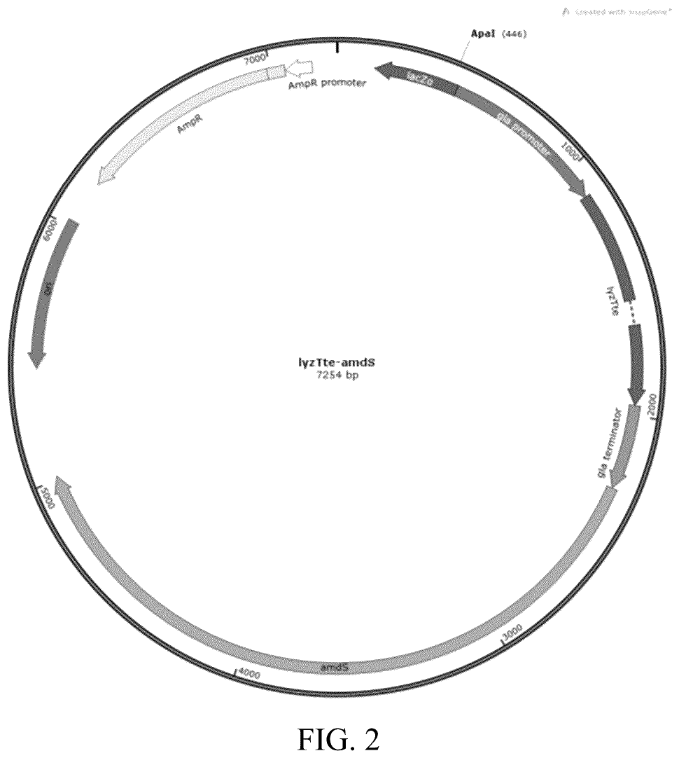

The lysozyme lyzTte expression plasmid was constructed as follows: primers lyzTte-F and lyzTte-R were used for PCR amplification to obtain a lyzTte gene with a recombinant arm, and then the lyzTte gene was recombined with the linearized pGla-amdS plasmid by using Gibson Master Mix Kit (E2611, New England Biolabs) to obtain a plyzTte-amdS plasmid, which was sequenced to confirm the sequences. The map of the constructed plasmid was as shown in FIG. 2. The plasmid could be linearized at an ApaI site and then used for protoplast transformation.

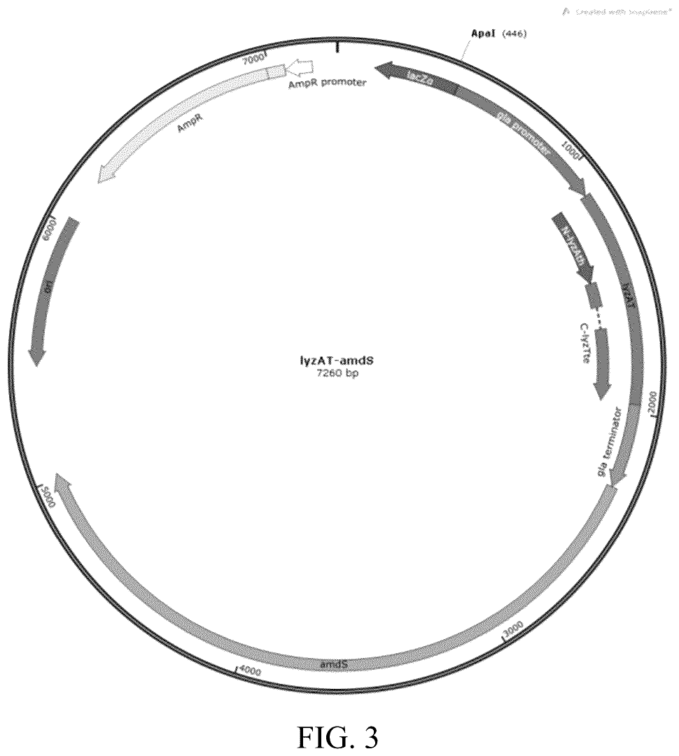

The lysozyme lyzAT expression plasmid was constructed as follows: primers lyzAth-F and lyzTte-R were used for PCR amplification to obtain a lyzAT gene with a recombinant arm, and then the lyzAT gene was recombined with the linearized pGla-amdS plasmid by using Gibson Master Mix Kit (E2611, New England Biolabs) to obtain a plyzAT-amdS plasmid, which was sequenced to confirm the sequences. The map of the constructed plasmid was as shown in FIG. 3. The plasmid could be linearized at an ApaI site and then used for protoplast transformation.

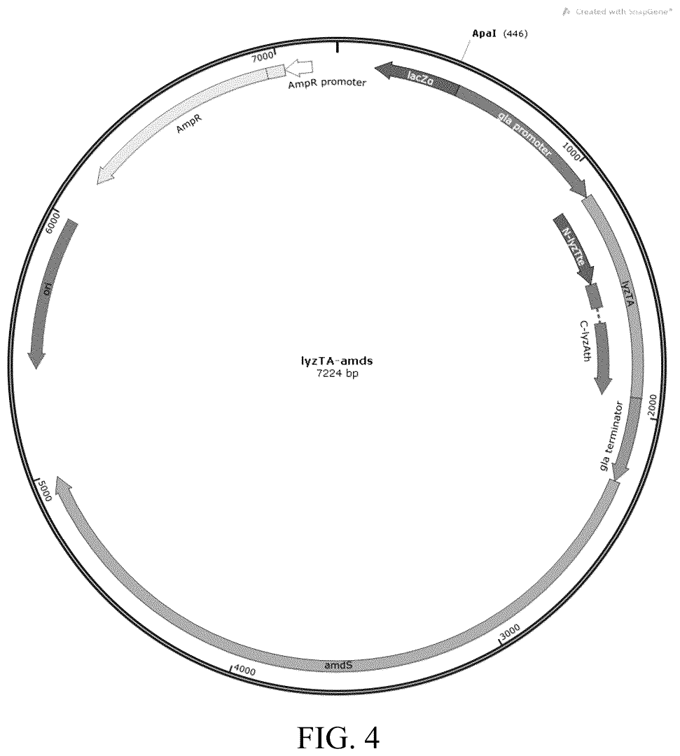

The lysozyme lyzTA expression plasmid was constructed as follows: primers lyzTte-F and lyzAth-R were used for PCR amplification to obtain a lyzTA gene with a recombinant arm, and then the lyzTA gene was recombined with the linearized pGla-amdS plasmid by using Gibson Master Mix Kit (E2611, New England Biolabs) to obtain a plyzTA-amdS plasmid, which was sequenced to confirm the sequences. The map of the constructed plasmid was as shown in FIG. 4. The plasmid could be linearized at an ApaI site and then used for protoplast transformation.

The relevant primer sequences were as follows:

TABLE 1
Primers in the present invention

| Primer | |
|---|---|
| name | Sequence (5′-3′) |
| vector-F | CTTGGCGTAATCATGGTCATAGC |
| | (SEQ ID NO: 9) |
| vector-R | CGGACCCCTCCGCCAATGGCCTTGCATGCAGGCCTCTGCA |
| | (SEQ ID NO: 10) |
| amdS-F | CTAGATCTACGCCAGGACCG |
| | (SEQ ID NO: 11) |
| amdS-R | ATGACCATGATTACGCCAAGCTTCTGGAAACGCAACCCTG |
| | (SEQ ID NO: 12) |
| gla-F | GCCATTGGCGGAGGGGTCCG (SEQ ID NO: 13) |
| gla-R | CGGTCCTGGCGTAGATCTAGATGCATTGAATGACAGTGAT |
| | (SEQ ID NO: 14) |
| lyzAth-F | catccccagcatcattacacctcagcaatgttgtaccttcc |
| | tctcgttgccc (SEQ ID NO: 15) |
| lyzAth-R | aggtgtcagtcaccctctagatctcgagctacagtccacca |
| | gcagcgcagatctt (SEQ ID NO: 16) |

lyzTte-F
(SEQ ID NO: 17)
catccccagcatcattacacctcagcaatgcagctctccctcctcgtc
lyzTte-R
(SEQ ID NO: 18)
gtgtcagtcaccctctagatctcgagttacaacccaccagcctggcaa
atct

### Example 2. Transformation and Integration of Lysozyme Expression Plasmids plyzAth-amdS, plyzTte-amdS, plyzAT-amdS and plyzTA-amdS

Four linearized lysozyme expression plasmids were introduced into strains of Aspergillus niger CICC2462 (purchased from China Center of Industrial Culture Collection, CICC) by using a protoplast transformation method, respectively, and the specific operation steps were as follows:
(1) Preparation of protoplasts: the mycelium of Aspergillus niger was inoculated in a nutritious TZ liquid culture medium (beef extract powder 0.8%, yeast extract 0.2%, peptone 0.5%, NaCl 0.2%, and sucrose 3%, pH 5.8), cultured for 48 h, and then filtered with Mira-cloth (Calbiochem company) to collect the mycelium, and washed with 0.7 M NaCl (pH 5.8); the washed mycelium was filtered to dryness, and then transferred to an enzymolysis solution (pH 5.8) containing cellulase 1% (Sigma), snail enzyme 1% (Sigma) and lywallzyme 0.2% (Sigma) for enzymolysis at 30° C. and 65 rpm for 3 h; subsequently, the enzymolysis solution containing protoplasts was placed on ice and filtered through four layers of lens paper; the resulting filtrate was gently centrifuged at 3000 rpm and 4° C. for 10 min, and then the supernatant was discarded; and the protoplasts attached to the tube wall were washed once with an STC solution (1 M D-Sorbitol, 50 mM CaCl₂, and 10 mM Tris, pH 7.5), and finally, the protoplasts were resuspended in an appropriate amount of the STC solution.
(2) Transformation of protoplasts: 10 μl (concentration: 1000 ng/μl) of the lysozyme expression plasmid linearized with ApaI was added to 100 μl of the protoplast suspension and mixed well; the mixture was placed at room temperature for 25 min, and then 900 μl of a PEG solution in total was added in three times and mixed well; the mixture was placed at room temperature for 25 min, and then centrifuged at room temperature at a rotating speed of 3000 rpm for 10 min, and the supernatant was discarded; the protoplasts attached to the tube wall were resuspended in 1 ml of the STC solution, mixed with an acetamide culture medium (sucrose 3%, KCl 0.05%, K₂HPO₄·3H₂O 0.1%, FeSO₄ 0.001%, MgSO₄ 0.0244%, acetamide 0.06%, and CsCl 0.34%) precooled to about 45° C., and spread on a plate; the plate was solidified, and then placed in a 34° C. incubator for 4-5 days; the transformants were picked into a new acetamide culture medium plate and placed in a 34° C. incubator for additional 4-5 days; and the grown transformants were called positive transformants.

Four linearized lysozyme expression plasmids lyzAth-amdS, lyzTte-amdS, lyzAT-amdS and lyzTA-amdS were transformed into strains of Aspergillus niger by using the above-mentioned protoplast transformation method, respectively, so as to obtain four lysozyme positive transformants.

### Example 3. Shake Flask Culture of Recombinant Expression Strain Formed by Lysozyme and Aspergillus niger

The four lysozyme positive transformants as described above were inoculated into a shake flask containing 50 ml of a YPM medium (yeast extract 0.2%, peptone 0.2%, and maltose 2%), respectively, and cultured on a shaker at a temperature of 34° C. and a rotating speed of 220 rpm for 5 days. The cultures were centrifuged to collect a supernatant of the fermentation broth, and the enzyme activity of the lysozyme was determined.

### Example 4. Determination of Lysozyme Activity

The lysozyme activity was determined according to the national standard GB/T 1886.257-2016.

Lysozyme can hydrolyze the cell wall of bacteria, leading to the lysis of Micrococcus luteus cell walls and consequently a decrease in the absorbance value of the solution. One unit of lysozyme activity is defined as the amount of lysozyme required to cause a change in absorbance of 0.001 per minute at 450 nm using a Micrococcus luteus suspension at 25° C. and pH 6.2.

Reagents and Materials

Micrococcus luteus: CICC10680 (purchased from China Center of Industrial Culture Collection, CICC).

0.1 mol/L phosphate buffer (pH 6.2).

11.70 g of sodium dihydrogen phosphate (NaH₂PO₄·2H₂O), 7.86 g of disodium hydrogen phosphate (Na₂HPO₄·12H₂O) and 0.372 g of disodium ethylenediamine tetra-acetic acid (EDTA-2Na) were weighed and added to sterile water, and the mixture was diluted to a constant volume of 1000 mL. The buffer solution was adjusted to pH 6.2±0.1.

Lysozyme standard: egg white lysozyme.

Substrate solution: 50 mL of Micrococcus luteus suspension was prepared from the phosphate buffer. Before use, the substrate was incubated at 37° C. for 30 min.

The substrate solution could be stable for 2 h at room temperature. The spectrophotometer was adjusted to zero point with the phosphate buffer, and then the absorbance of the substrate solution was determined. The reading at 450 nm should be 0.70±0.1.

Preparation of Standard Solution:

50 mg of egg white lysozyme standard was accurately weighed and added into a 50 mL volumetric flask, and dissolved in about 25 mL of the phosphate buffer with stirring. The mixture was diluted to a constant volume, and mixed thoroughly (if necessary, the solution was frozen for subsequent determination). 3 mL of the above standard preparation solution was transferred to a 100 mL volumetric flask, and dissolved in the phosphate buffer with stirring, and the mixture was diluted to a constant volume.

Determination:

3 standard solutions and 3 sample solutions were taken for determination. At room temperature of 25° C., a 1 cm cuvette was put into a spectrophotometer, and the spectrophotometer was adjusted to zero absorbance with the phosphate buffer. 2.9 mL of substrate solution was pipetted into the cuvette, and the initial absorbance at 450 nm should be 0.70±0.10. The determination could only be started when the change in the initial absorbance value within 3 min was less than or equal to 0.003. 0.1 mL of standard solution was pipetted into the substrate solution and mixed thoroughly. Changes in the absorbance value within 3 min were recorded, and the absorbance value was recorded every 15 s. The changes in the absorbance value per minute should be within 0.03-0.08, and if it went beyond this required range, the concentration of the sample solution should be adjusted. The operation was repeated to determine the sample solution. The reading in the first 1 min should be omitted in the calculation as the reaction became stable after 1 min.

Result Calculation

The enzyme activity X was calculated according to equation (A.1):

$$X = \frac{( {A}_{1} - {A}_{2} )}{2 \times m \times 0.001} \qquad ( A \text{.1} )$$

In the equation:
A₁: the absorbance of the sample at 450 nm upon 1 min of reaction;
A₂: the absorbance of the sample at 450 nm upon 3 min of reaction;
m: the mass of lysozyme in the sample solution used for analysis, expressed in milligram (mg);
2: the time taken to obtain absorbance readings at 1 min and 3 min, expressed in minutes (min);
0.001: the value of the decrease in absorbance per minute caused by one unit of lysozyme.

### Example 5. Determination of Heat Resistance of Lysozyme

The lysozyme sample to be tested was diluted to 5000 U/mL using the phosphate buffer. 4.5 mL of the phosphate buffer was added into a test tube, and preheated for 5 min at different temperatures. 0.5 mL of diluent was added to the test tube, and mixed evenly. The mixture was treated in water bath at 60° C. for 3 min, and taken out and then cooled down to room temperature in ice water bath. The cooled mixture was determined for enzyme activity according to the lysozyme activity determination method. Calculation: the residual activity of heat-resistance enzyme as percent of the initial activity (%)=U_{X}/U₀*100%, where U₀ is the enzyme activity before water bath treatment, and U_{X} is the enzyme activity after water bath treatment. The determination results were as shown in FIG. 4. The residual activity of Lysozyme lyzAth derived from Aspergillus thermomutatus and lysozyme lyzTte derived from Thermothielavioides terrestris were 8% and 26%, respectively, after being treated in water bath at 60° C. for 3 min; while the constructed hybrid lysozyme lyzAT is 60% under the same conditions, suggesting that the heat resistance was greatly improved compared to a parental sequence; further, the residual activity of the constructed hybrid lysozyme lyzTA is only 25% under the same conditions, which was close to the heat resistance of the parental lysozyme lyzTte.

TABLE 2
Determination of residual heat-resistance enzyme activity of lysozyme

| | | | Residual activity of heat- |
|---|---|---|---|
| | | | resistance enzyme as percent of |
| Lysozyme | U₀ (U/mL) | U_{X} (U/mL) | the initial activity (%) |
| lyzAth | 53710 | 4250 | 8 |
| lyzTte | 101310 | 26140 | 26 |
| lyzAT | 220000 | 131220 | 60 |
| lyzTA | 138120 | 35110 | 25 |

### Example 6

1. Test Materials

Test animals: Arbor Acres (AA) male broilers.

Enzyme for test: lysozyme lyzAT (500,000 U/g, detected by the national standard method) having an amino acid sequence set forth in SEQ ID NO: 8.

2. Test Design

700 1-day-old healthy Arbor Acres (AA) male broilers were selected and randomly divided into 5 treatment groups, with no significant difference in average weight among the treatment groups. 14 replicates were set for each treatment group, with 10 broilers in each replicate. The 5 treatment groups were divided into 1 control group and 4 test groups, and the addition amounts of lysozyme lyzAT in the control group and each test group were as follows, respectively: 0, 250 g/t, 500 g/t, 1000 g/t and 3000 g/t.

The test period lasted for 42 days in total, and consisted of two stages: early stage and late stage.

3. Test Diet and Nutritional Level

In this test, corn-soybean meal basal diet was selected and formulated separately according to 2 stages of the 0-21-day-old stage and the 22-42-day-old stage as guided by Nutritional Requirements of Chicken of NRC (1994), Feeding Standard of Chicken in China (2004) and Tables of Feed Composition and Nutritive Values in China (2020). The composition and nutritional level of the basal diet were as shown in Table 3.

TABLE 3
Formula and nutritional level % (air-dried basis) of basal diet

| Item | 0-21-day-old | Item | 22-42-day-old |
|---|---|---|---|
| Raw material | | Raw material | |
| Corn | 56.76 | Corn | 57.92 |
| Dehulled soybean | 26.5 | Dehulled soybean | 26 |
| meal | | meal | |
| Corn gluten meal | 3.2 | Corn gluten meal | 3 |
| Fine stone powder | 1.1 | Fine stone powder | 1.1 |
| Calcium hydrogen | 1.1 | Calcium hydrogen | 1.1 |
| phosphate | | phosphate | |
| Chicken oil | 6.5 | Chicken oil | 6 |
| Glutamic acid | 2 | Glutamic acid | 2 |
| residue | | residue | |
| Feather meal | 0.5 | Feather meal | 0.5 |
| Sodium chloride | 0.28 | Sodium chloride | 0.28 |
| Lysine 70% | 0.78 | Lysine 70% | 0.77 |
| Methionine 99% | 0.22 | Threonine 98.5% | 0.1 |
| Enzymemate B | 0.05 | Methionine 99% | 0.2 |
| Glucose oxidase | 0.01 | Enzymemate B | 0.02 |
| Premix 1) | 1 | Glucose oxidase | 0.01 |
| Premix 1) | 1 | | |
| Total | 100 | Total | 100 |
| Nutritional level | | | |
| Metabolic energy | 11.42 | | 13.49 |
| (MJ/kg) 2) | | | |
| Crude protein | 20.0 | | 20.5 |
| Crude fat | 4.21 | | 5.51 |
| Calcium | 0.97 | | 0.77 |
| Phosphorus | 0.41 | | 0.32 |

Notes:
in Table 3:
1) Premix provides the following per kilogram of diet: VD₃ 2 750 IU, VE 20 IU, VK₃ 2 mg, VB₁ 1.5 mg, VB₂ 6 mg, pantothenic acid 12 mg, nicotinic acid 20 mg, VB₆ 2.5 mg, VB₁₂ 2.03 mg, Mn 75 mg, Zn 75 mg, Fe 95 mg, Cu 10 mg, I 0.6 mg, and Se 0.3 mg.
2) Metabolic energy is a calculated value, and the rest is a measured value.
4. Feeding management

The test was carried out in the experimental chicken farm of Shenyang Agricultural University. Before the test, the surrounding environment, chicken houses and tools to be used were disinfected. Chickens to be tested were raised in three-dimensional cages, and 14 replicates in each group were arranged separately according to the different positions of the cages, so that the test conditions of each group remained the same except for the test parameters of different lysozyme products and doses. Chickens were given free access to food and water, and raised under free ventilation and all-day illumination. Feeding management and immunization were carried out according to normal procedures, and data collection and recording were made on time.

5. Determination Indicators

5.1 Production Performance

During the feeding test, the feed given and the feed consumption were recorded every day, and broilers in each replicate were weighed on an empty stomach on d1, d21 and d42 of the test, so as to calculate the average daily feed intake (ADFI), average daily gain (ADG) and feed-to-gain ratio (F/G) in each stage and the whole growth period. Death cases were observed and recorded carefully every day, so as to calculate the mortality.

The average daily feed intake, average daily gain, feed-to-gain ratio and mortality are calculated as follows:

average daily feed intake (ADFI)=feed consumption in each treatment group during the test period/(test days×number of broilers in each treatment group);

average daily gain (ADG)=weight gain in each treatment group during the test period/(test days×number of broilers in each treatment group);

feed-to-gain ratio (F/G)=average daily feed intake/average daily gain;

mortality (%)=number of mortal broilers at the end of the test/number of broilers at the beginning of the test×100%.

5.2 European Index

The European index is a comprehensive evaluation of the weight, survival rate, feed-to-gain ratio, production management and other indicators of broilers, which reflects the level of profitability. The larger the index, the more profitable it is.

The European index is calculated as follows:

European index=survival rate×weight (kg)/(feed-to-meat ratio×days to market)×10000.

5.3 Calculation of Economic Benefit Indicator

The comprehensive economic benefit is equal to the sales price of broilers minus the production cost of broilers, in which the main factors affecting the production cost of broilers are the feed-to-meat ratio, the average feed price and the cost of baby chicks; in addition, the market price of broilers when they are marketed also has a great influence on the economic benefit of broiler feeding.

According to the feed consumption, growth conditions and other data, the comprehensive economic benefits of the control group and the test groups were compared.

5.4 Data Processing and Statistical Analysis

Recording and arrangement of test data: EXCEL software was used for data processing and analysis, and SPSS 17.0 software was used for one-way ANOVA. When the differences were significant, a Dun-can method was used for multiple comparison, with P<0.05 as the significant level and P<0.01 as the extremely significant level, and the results were expressed as “mean±standard deviation”.

### Results and Analysis

1. Effect of Lysozyme lyzAT on Production Performance of Broilers

As could be seen from the Table 4, at the 0-21-day-old stage, the final weight of broilers in test groups of lysozyme lyzAT 500 g/t and 1000 g/t was significantly higher than that in the control group (P<0.05), but there was no significant difference between the remaining test groups and the control group (P>0.05); the average daily gain of broilers in test groups of lysozyme lyzAT 500 g/t and 1000 g/t was significantly higher than that in the control group (P<0.05), but there was no significant difference between the remaining test groups and the control group (P>0.05); with respect to the feed-to-gain ratio, test groups all showed a decreasing trend compared with the control group (P=0.089), with a decrease of 1.55% in test groups of lysozyme lyzAT 250 g/t, 500 g/t and 1000 g/t; and there was no significant difference in the average daily feed intake between test groups and the control group (P>0.05).

At the 22-42-day-old stage, the final weight of broilers in the test group of lysozyme lyzAT 250 g/t was extremely significantly higher than that in the control group (P<0.01), and the final weight of broilers in the test group of lysozyme lyzAT 500 g/t was significantly higher than that in the control group (P<0.05), but there was no significant difference between the remaining test groups and the control group (P>0.05); the average daily gain in test groups of lysozyme lyzAT 250 g/t and 500 g/t was extremely significantly higher than that in the control group (P<0.01), and the average daily gain in the test group of 1000 g/t was significantly higher than that in the control group (P<0.05), but there was no significant difference between the test group of 3000 g/t and the control group (P>0.05); the feed-to-gain ratio in test groups of lysozyme lyzAT 250 g/t and 500 g/t was significantly lower than that in the control group (P<0.05), but there was no significant difference between the remaining test groups and the control group (P>0.05); and with respect to the average daily feed intake, there was an increasing trend in test groups compared with the control group (P=0.075), and the test group of lysozyme lyzAT 500 g/t showed an increase of 2.50% compared with the control group.

At the whole 0-42-day-old stage, the average daily gain in test groups of lysozyme lyzAT 250 g/t, 500 g/t and 1000 g/t was all extremely significantly higher than that in the control group (P<0.01); the feed-to-gain ratio in the test group of lysozyme lyzAT 250 g/t was extremely significantly lower than that in the control group (P<0.01), and the feed-to-gain ratio in the test group of lysozyme lyzAT 500 g/t was significantly lower than that in the control group (P<0.05); and there was no significant difference in the average daily feed intake among the test groups (P>0.05).

TABLE 4
Effect of lysozyme lyzAT on production performance of broilers

| | | Treatment group | | | | | |
|---|---|---|---|---|---|---|---|
| | Age in | Control | 250 | 500 | 1000 | 3000 | P |
| Item | days | group | g/t | g/t | g/t | g/t | value |
| Weight | 0 | 38.00 | 38.00 | 38.00 | 38.00 | 38.00 | |
| (g) | 21 | 855.35 ± | 857.85 ± | 862.14 ± | 862.85 ± | 860.35 ± | 0.040 |
| | | 7.19^{b} | 6.71^{ab} | 6.99^{a} | 8.01^{a} | 6.64^{ab} | |
| | 42 | 2558.92 ± | 2708.64 ± | 2632.11 ± | 2596.45 ± | 2616.07 ± | 0.000 |
| | | 53.12^{Bc} | 83.16^{Aa} | 60.30^{Bb} | 70.43^{Bbc} | 73.41^{Bbc} | |
| Average | 0-21 | 38.91 ± | 39.03 ± | 39.24 ± | 39.27 ± | 39.15 ± | 0.039 |
| daily gain | | 0.34^{b} | 0.31^{ab} | 0.33^{a} | 0.38^{a} | 0.31^{ab} | |
| (g) | 22-42 | 81.19 ± | 87.69 ± | 87.29 ± | 84.73 ± | 83.18 ± | 0.000 |
| | | 2.69^{Cc} | 3.91^{Aa} | 4.66^{ABa} | 5.05^{ABCab} | 3.32^{BCbc} | |
| | 0-42 | 59.80 ± | 63.58 ± | 63.27 ± | 62.05 ± | 61.38 ± | 0.000 |
| | | 1.31^{Cc} | 1.98^{Aa} | 2.38^{ABa} | 12.50^{ABab} | 1.74^{BCb} | |
| Average | 0-21 | 50.19 ± | 49.82 ± | 49.93 ± | 50.16 ± | 50.12 ± | 0.261 |
| daily feed | | 0.61 | 0.51 | 0.47 | 0.45 | 0.49 | |
| intake (g) | 22-42 | 124.95 ± | 126.19 ± | 128.07 ± | 126.91 ± | 125.72 ± | 0.075 |
| | | 2.21 | 2.56 | 3.41 | 3.53 | 2.96 | |
| | 0-42 | 87.57 ± | 88.29 ± | 89.01 ± | 88.53 ± | 87.92 ± | 0.155 |
| | | 1.17 | 1.57 | 1.74 | 1.86 | 1.43 | |
| Feed-to- | 0-21 | 1.29 ± | 1.27 ± | 1.27 ± | 1.27 ± | 1.28 ± | 0.089 |
| gain ratio | | .0.02 | 0.01 | 0.01 | 0.02 | 0.02 | |
| | 22-42 | 1.54 ± | 1.44 ± | 1.47 ± | 1.50 ± | 1.51 ± | 0.021 |
| | | 0.05^{a} | 0.07^{c} | 0.09^{bc} | 0.11^{abc} | 0.05^{ab} | |
| | 0-42 | 1.45 ± | 1.38 ± | 1.40 ± | 1.43 ± | 1.43 ± | 0.007 |
| | | 0.03^{Aa} | 0.04^{Bc} | 0.06^{ABbc} | 0.07^{ABab} | 0.03^{ABab} | |

Notes:
different lowercase letters occurring as the superscript of data in the same row indicate significant differences (P < 0.05); capital letters indicate extremely significant differences (P < 0.01); and the same letters or no letters indicate no significant differences (P > 0.05).

2. Effect of Lysozyme lyzAT on European Index of Broilers

As could be seen from Table 5, the European index of the control group was the lowest compared with all the test groups throughout the test, and the European index of the test group of lysozyme lyzAT 250 g/t was the highest throughout the test, which was 62.58 higher than that of the control group, indicating that adding lysozyme can improve the profitability of broilers.

TABLE 5
Effect of lysozyme lyzAT on European index of broilers

| | Treatment group | | | | |
|---|---|---|---|---|---|
| Item | Control group | 250 g/t | 500 g/t | 1000 g/t | 3000 g/t |
| Lysozyme A | 385.50 | 448.08 | 422.96 | 412.70 | 407.21 |

3. Effect of Lysozyme lyzAT on Breeding Benefit of Broilers

The price of chicks is 1.95 yuan/chick; the price of commercial live chickens is 8.4 yuan/kg; and the price of feeds is 3,500 yuan/t. As could be seen from Table 6, the average profit per chicken of all test groups of lysozyme lyzAT was higher than that of the control group, with the average profit per chicken of the test group of lysozyme lyzAT 250 g/t being the highest, which was 1.08 yuan higher than that of the control group.

TABLE 6
Effect of lysozyme lyzAT on breeding benefit of broilers

| | | Treatment group | | | | |
|---|---|---|---|---|---|---|
| | | Control | 250 | 500 | 1000 | 3000 |
| Item | Age in days | group | g/t | g/t | g/t | g/t |
| Lysozyme | Average weight gain per | 2.52 | 2.67 | 2.59 | 2.56 | 2.58 |
| lyzAT | chicken (kg) | | | | | |
| | Average weight gain benefit | 21.17 | 22.43 | 21.76 | 21.50 | 21.67 |
| | per chicken (yuan) | | | | | |
| | Average feed consumption per | 12.52 | 12.71 | 12.42 | 12.45 | 12.64 |
| | chicken (yuan) | | | | | |
| | Average profit per chicken | 6.69 | 777 | 7.39 | 7.10 | 7.08 |
| | (yuan) | | | | | |

Therefore, it can be seen from the above test data that the feed with lysozyme lyzAT added can significantly improve the weight, average daily gain and average daily feed intake of broilers at all stages, and at the same time can significantly reduce the feed-to-gain ratio. Additionally, the remarkable improvement of the European index and economic benefit indicators also shows that the breeding benefit of lysozyme lyzAT is rather considerable, and the addition amount of the lysozyme can be 100-1000 g/t, with the most suitable addition amount being 250-500 g/t.

The content of protection of the present invention is not limited to the above examples. Without departing from the spirit and scope of the present invention, variations and advantages that can be conceived by those skilled in the art are all included in the present invention, and the scope of protection shall be in accordance with the appended claims.

1. A chimeric lysozyme variant, wherein the chimeric lysozyme variant has an amino acid sequence set forth in SEQ ID NO: 8, or having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 8; and the chimeric lysozyme variant has a lysozyme activity.

2. The chimeric lysozyme variant of claim 1, wherein the chimeric lysozyme variant has an amino acid sequence set forth in SEQ ID NO: 8.

3. Use of the chimeric lysozyme variant of claim 1 in the preparation of a product as an alternative to an antibiotic.

4. A polynucleotide, wherein the polynucleotide encodes the chimeric lysozyme variant of claim 1.

5. The polynucleotide of claim 4, wherein the polynucleotide has a nucleotide sequence set forth in SEQ ID NO: 7.

6. A recombinant vector, wherein the recombinant vector contains the polynucleotide of claim 4.

7. A host cell, wherein the host cell contains the recombinant vector of claim 6.

8. The host cell of claim 7, wherein the host cell is selected from any of the Aspergillus niger, Pichia pastoris, Aspergillus oryzae, Trichoderma reesei, Bacillus subtilis, Bacillus licheniformis or Escherichia coli.

9. Use of the chimeric lysozyme variant of claim 1, wherein the use is:
in an animal feed;
in an animal feed additive;
in the preparation of a composition for use in an animal feed;
for improving the nutritional value of an animal feed;
for improving the digestibility of an animal feed; and/or
for improving one or more performance parameters of an animal.

10. An animal feed additive, wherein the animal feed additive comprises the chimeric lysozyme variant of claim 1.

11. The animal feed additive of claim 10, wherein the chimeric lysozyme variant is added in an amount of 100-1000 g chimeric lysozyme variant per ton of animal feed; preferably, the chimeric lysozyme variant is added in an amount of 250-500 g chimeric lysozyme variant per ton of animal feed.
